(19) United States
(12) Patent Application Publication
(10) Pub. No.: US 2026/0031209 A1
(43) Pub. Date: Jan. 29, 2026

(54) APPARATUS AND METHOD FOR MONITORING COMPLIANCE WITH PATIENT CARE PROTOCOLS

(71) Applicant: Invisalert Solutions, Inc., West Chester, PA (US)

(72) Inventors: Peter A. NAGY, Newtown Square, PA (US); Christopher DUNN, West Chester, PA (US); Mary Kay SHIBLEY, West Chester, PA (US)

(21) Appl. No.: 18/955,963

(22) Filed: Nov. 21, 2024

Related U.S. Application Data
(60) Provisional application No. 63/676,307, filed on Jul. 26, 2024.

(51) Int. Cl. G16H 20/00; G16H 10/60; G16H 15/00; G16H 40/20

(52) U.S. Cl. CPC G16H 20/00; G16H 10/60; G16H 15/00; G16H 40/20

(57) ABSTRACT

Apparatuses and methods for monitoring and verifying the performance of one or more patient health care protocols may identify appropriate patient protocols, organize patient protocols across multiple patients for specific caregivers and/or hospital units, display and/or order patient protocols, and/or track compliance with the patient protocols. Protocols may be determined from patient healthcare records and delivered to healthcare staff. The healthcare staff can perform or execute the patient care protocols and the apparatus or method may automatically and non-obtrusively collect evidence that the care protocol has been performed. The patient's healthcare records can be updated to include care protocol verification information.

### CLAIM OF PRIORITY

This patent application claims priority to U.S. Provisional Patent Application No. 63/676,307, titled “APPARATUS AND METHOD FOR MONITORING COMPLIANCE WITH PATIENT CARE PROTOCOLS,” filed on Jul. 26, 2024, and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are apparatuses (e.g., devices, systems, etc.) that may be used to monitor compliance with patient care protocols a healthcare setting.

### BACKGROUND

Within medical care settings, a wide range of patient care “protocols” or “pathways” have been developed to minimize poor patient outcomes, length of stay, and procedural complications. A protocol generally can refer to steps or operations that are used to care for or treat particular patient illnesses or conditions. For example, when a central venous catheter or “central line” is inserted into a patient, most hospitals will aspire to follow a specific protocol to prevent central line-associated blood stream infection (CLABSI). A CLABSI protocol might have ten or more steps for caregivers to follow such as inspecting the insertion site for infection, cleaning or replacing lines, among others. In some healthcare settings, monitoring the performance of care protocols can be difficult. Often, there are no compliance mechanisms, documentation of the performance of the step of a protocol is manual and error prone, and protocol workflow can be inefficient.

Thus, there exists a need for an accurate and easy to use protocol compliance monitoring system.

### SUMMARY OF THE DISCLOSURE

Described herein are apparatuses (e.g., systems and devices, including software, hardware and firmware), and methods to manage and track compliance with health care protocols, particularly within a healthcare setting such as a hospital, residential treatment centers, and the like. In general, a computer-implemented method is used to determine and assign aspects of any number of patient care protocols to a patient. The method can include determining the location of the patient with respect to a caregiver, and prioritizing the activities of the caregivers to help ensure the performance and the documentation of care protocols.

In some examples, the location of patients and caregivers may be determined through wireless signal characteristics associated with wireless devices worn by or held by the patients or the caregivers. The computer-implemented method uses patient and caregiver locations to alert particular caregivers near particular patients to perform one or more compliance steps.

Methods described herein can verify the performance of patient care protocols and can include displaying, on a user interface of a mobile device, one or more elements of a patient care protocol, capturing, with the mobile device, evidence of performance of the one or more elements of the patient care protocol, transmitting, to a computing device, the evidence of performance, and updating electronic health records to indicate compliance with the patient care protocol.

For example, described herein are methods comprising: receiving or accessing, in one or more processors, one or more hospital or physician orders for each patient of a set of patients within a healthcare location; determining, by the one or more processors, a patient care protocol for each of the hospital or physician orders for each patient, wherein each patient care protocol comprises: one or more steps to be performed by a caregiver, one or more timers associated with the one or more steps, and one or more compliance indicators for each of the one or more steps, wherein the compliance indicators each include a proximity indicator and/or a visual indicator; displaying, on a user interface, a time-ordered list of the patient care protocols for the set of patients, with a countdown time associated with each patient care protocol in the time-ordered list; detecting proximity data between the caregiver and a patient having a patient care protocol in the time-ordered list; confirming compliance with each step of the patient care protocol if the time-ordered list of patient care protocols based on receipt of the proximity indicator and/or a visual indicator by the processor, the proximity indicator associated with the patient care protocol and the one or more times associated with the one or more steps; and outputting an indication that all or some of the one or more steps of the patient care protocol have been completed based on the confirmed compliance.

In some cases a method may include: receiving or accessing one or more physician orders for each patient of a set of patients within a healthcare location; identifying a patient care protocol associated with each patient of the set of patients from the physician orders, wherein each patient care protocol comprises one or more steps to be performed by a caregiver, one or more times associated with the one or more steps, and a proximity indicator associated with the one or more steps of the patient care protocol; displaying, on a user interface, a time-ordered list of the patient care protocols for the set of patients, with a countdown time associated with each patient care protocol in the time-ordered list; detecting proximity data between the caregiver and a patient having a patient care protocol in the time-ordered list; confirming compliance with a patient care protocol of the time-ordered list of patient care protocols based on the proximity data, the proximity indicator associated with the patient care protocol and the one or more times associated with the one or more steps; and outputting an indication that all or some of the one or more steps of the patient care protocol have been completed based on the confirmed compliance.

Any of these methods and apparatuses may include detecting proximity data. Proximity may refer to proximity between the caregiver and the patient. Proximity data may be detected directly (e.g., directly from a radar—or other detection modality that detects the relative position between a patient and the caregiver), comprises detecting proximity data using a mobile device held by the caregiver. For example, detecting proximity data may comprise detecting proximity using one or more of: a Light Detection and Ranging (LiDAR) signal, a radar signal, an ultrasound signal and/or a radio frequency (RF) signal between a caregiver device and a patient worn device. The RF signal may comprise an RF received signal strength indicator (RSSI) from directly between devices carried by the caregiver and the patient. In some cases the RF signal between the caregiver device and the patient worn device comprise one of: an RF angle of arrival (AoA) and/or a time of flight scheme, from a caregiver device and a patient-worn device. The LiDAR, radar or ultrasound may create a two- or three-dimensional physical representation model to gauge proximity by comparing temporal changes in the model based on distance and/or motion.

In general, these methods and apparatuses may include (e.g., when identifying the patient care protocol associated with each patient of the set of patients from the physician orders) calculating the one or more times associated with the one or more steps of each patient care protocol. In some cases a single time may be associated; in other cases multiple times (e.g., corresponding to multiple and/or repeated steps) may be included.

Any of these methods and apparatuses may capture compliance with patient physical exercises, such as, but not limited to, patient ambulation distance (ERAS), and automatically capture that data. For example, the methods and apparatuses may capture proximity and visual data from the patient, and may use this information to determine physical exercise data. This may be another potential compliance indicator, including distance traveled. These methods and apparatuses may also include one or more microphones which may provide another compliance indication based on sound.

In general, any of these methods and apparatuses may include detecting proximity data by detecting proximity using one or more of: a LiDAR signal, a radar signal, an ultrasound signal and/or a radio frequency (RF) signal between a caregiver device and a patient worn device. RF data can be determined between devices. Radar, etc. can detect the caregiver and patient position and therefore proximity without the need for a device worn/held by the caregiver and/or patient. In the methods and apparatuses described herein a combination of radar and device interaction (RF) may both be used. Any of these methods and apparatuses may include an integrated Bluetooth device as part of the hardware that may also include a radar sensor with a backhaul, as described herein. In some cases the radar may detect the location of a caregiver and/or a patient and thereby proximity but this can be done by an integrated Bluetooth receiver via RSSI or AoA, AoD.

Each patient protocol may include one or more compliance indicators associated with the one or more steps. A compliance indicator may include the proximity data and/or it may include other indicators associated with a particular patient care protocol (e.g., entry of patient data, entering drug dispensing/use data, etc.). In any of these methods and apparatuses, confirming compliance with the patient care protocol of the time-ordered list of patient care protocols may be based on the proximity data, the proximity indicator associated with the patient care protocol, the one or more times associated with the one or more steps and the one or more compliance indicators.

In any of these methods and apparatuses, identifying the patient care protocol may comprise identifying the patient care protocol from a database of care protocols. This database may be proctored and maintained by a third party, which may be separately updated and/or accessed. In some cases the patient care protocols may be health care provider (doctor, nurse, hospital, clinic, etc.) specific.

Receiving or accessing the one or more physician orders for each patient of a set of patients within a healthcare location may include receiving or accessing in a processor. The processor may include or access patient electronic health records (EHR), or other healthcare provider specific systems.

Any of these methods and apparatuses may include identifying the patients within the healthcare location. A set of patients may include one patient, two patients, 3 patients, 4 patients, 5 patients, etc. (e.g., between 1-30, 1-25, 1-20, 1-15, 1-10, etc. patients). The set of patients may be specific to a particular caregiver (nurse, doctor, etc.) and/or to a specific portion of a healthcare location (e.g., unit, floor, hall, etc.). Patients may be identified by name, patient ID number and/or location (bed, room, etc.).

Displaying may include displaying the time-ordered list of the patient care protocols for the set of patients on a user interface of a mobile device, e.g., a caregiver mobile device (e.g., phone, watch, tablet, etc.). The same display or a portion of the display or an aggregate display (for multiple caregivers/locations) may be displayed in a master display, e.g., an administrator or supervisory display.

Outputting the indication that all or some of the one or more steps of the patient care protocol have been completed may include transmitting to a remote device. Outputting may include storing, transmitting and/or displaying. In any of these methods, outputting may comprise removing a completed patient care protocol from the time-ordered list of the patient care protocols. In general, these methods may include updating the user interface of the caregiver device to reflect partial and/or complete completion of the patient care protocol.

For example, a method may include: receiving or accessing, in a processor, one or more physician orders for each patient of a set of patients within a healthcare location; identifying, from a database of care protocols, a patient care protocol associated with each patient of the set of patients from the physician orders, wherein each patient care protocol comprises one or more steps to be performed by a caregiver, one or more times associated with the one or more steps, and a proximity indicator associated with the one or more steps of the patient care protocol; displaying, on a user interface of a caregiver device, a time-ordered list of the patient care protocols for the set of patients, with a countdown time associated with each patient care protocol in the time-ordered list; detecting proximity data between the caregiver and a patient having a patient care protocol in the time-ordered list; confirming compliance with a patient care protocol of the time-ordered list of patient care protocols based on the proximity data, the proximity indicator associated with the patient care protocol and the one or more times associated with the one or more steps; and outputting an indication that all or some of the one or more steps of the patient care protocol have been completed based on the confirmed compliance and updating the user interface of the caregiver device to reflect partial and/or complete completion of the patient care protocol.

Also described herein are apparatuses (e.g., systems, software and/or hardware) for performing any of these methods. In general, a system may include: one or more processors and a memory coupled to the one or more processors, the memory storing computer-program instructions, that, when executed by the one or more processors, perform a computer-implemented method comprising any of the steps described above. For example, a system may include: a memory coupled to the one or more processors, the memory storing computer-program instructions, that, when executed by the one or more processors, perform a computer-implemented method comprising: receiving or accessing one or more physician orders for each patient of a set of patients within a healthcare location; identifying a patient care protocol associated with each patient of the set of patients from the physician orders, wherein each patient care protocol comprises one or more steps to be performed by a caregiver, one or more times associated with the one or more steps, and a proximity indicator associated with the one or more steps of the patient care protocol; displaying, on a user interface, a time-ordered list of the patient care protocols for the set of patients, with a countdown time associated with each patient care protocol in the time-ordered list; detecting proximity data between the caregiver and a patient having a patient care protocol in the time-ordered list; confirming compliance with a patient care protocol of the time-ordered list of patient care protocols based on the proximity data, the proximity indicator associated with the patient care protocol and the one or more times associated with the one or more steps; and outputting an indication that all or some of the one or more steps of the patient care protocol have been completed based on the confirmed compliance.

In some examples the system includes: one or more processors; and a memory coupled to the one or more processors, the memory storing computer-program instructions, that, when executed by the one or more processors, perform a computer-implemented method comprising: receiving or accessing, in a processor, one or more physician orders for each patient of a set of patients within a healthcare location; identifying, from a database of care protocols, a patient care protocol associated with each patient of the set of patients from the physician orders, wherein each patient care protocol comprises one or more steps to be performed by a caregiver, one or more times associated with the one or more steps, and a proximity indicator associated with the one or more steps of the patient care protocol; displaying, on a user interface of a caregiver device, a time-ordered list of the patient care protocols for the set of patients, with a countdown time associated with each patient care protocol in the time-ordered list; detecting proximity data between the caregiver and a patient having a patient care protocol in the time-ordered list; confirming compliance with a patient care protocol of the time-ordered list of patient care protocols based on the proximity data, the proximity indicator associated with the patient care protocol and the one or more times associated with the one or more steps; and outputting an indication that all or some of the one or more steps of the patient care protocol have been completed based on the confirmed compliance and updating the user interface of the caregiver device to reflect partial and/or complete completion of the patient care protocol.

Also described herein is software configured to perform any of these methods including non-transitory computing device readable medium having instructions stored thereon that are executable by a processor to cause a computing device to perform any of the methods described herein.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a flowchart of a method for generating or creating patient care protocols and distributing the care protocols to caregiving staff.

FIG. 2 shows an example user interface and flowchart for establishing or building care protocols for any number of patients.

FIG. 3 shows an example care protocol prioritization method and associated user interface.

FIG. 4 shows various user interfaces to alert caregivers regarding the performance of care protocols.

FIG. 5 is a flowchart showing an example method for workflow for performing and verifying patient care protocols.

FIG. 6 shows an example system for monitoring compliance with patient care protocols.

FIG. 7 shows a system that can wholly or partially be included in an apparatus for monitoring patient care protocols, similar to that shown in FIG. 6.

FIG. 8 shows a system that can wholly or partially be included in an apparatus for monitoring patient care protocols, similar to that shown in FIG. 6.

FIG. 9A shows a system that can wholly or partially be included in an apparatus for monitoring patient care protocols, similar to that shown in FIG. 6.

FIG. 9B shows an example of a subsystem that can be part of an apparatus for monitoring compliance with patient care protocols.

FIG. 10 shows a block diagram of a device that may perform any of the methods or procedures described herein.

FIG. 11 shows an example of a subsystem that can be part of an apparatus for monitoring compliance with patient care protocols; in FIG. 11 the subsystem is configured to precisely measure distance or ambulation.

FIG. 12 shows an example of a subsystem that can be part of an apparatus for monitoring compliance with patient care protocols; FIG. 12 further indicates the use of AoA or AoD technology to measure distance traveled by a patient or caregiver.

### DETAILED DESCRIPTION

Managing and tracking the performance of patient care protocols can be laborious and difficult. In a healthcare setting, such as a hospital (by way of example), the number of patients multiplied by the number of care protocols that can be associated with each patient can become unwieldy. Care protocols, sometimes called “bundles,” describe various preventative activities that should be performed with patients to treat patient conditions. In addition, care protocols may be associated with a performance frequency (hourly, daily, shift, etc.). Apparatuses and methods described herein can be used to assemble and track the performance of various care protocols for any feasible number of patients. In some examples, a caregiver can be presented with a prioritized list of patients with their associated care protocols to be performed. The list can be prioritized with respect to scheduled performance time.

The present disclosure is related to apparatuses (devices, systems, and the like), methods, and computing device readable media, which solve technical problems related to monitoring and verifying the performance of health care protocols for patients, particularly within healthcare settings. In some examples, care protocol information can be delivered to healthcare staff and alerts provided to remind the staff to perform one or more care protocols for one or more patients. Performance of the care protocol can be verified and a verification report can be generated.

In general, any of the systems, devices, methods, etc. described herein can locate, track, and verify performance of patient care protocols. In some examples, the apparatuses and/or methods can determine a location of caregivers and patients. The location information can be used to provide reminders (with respect to care protocols) to caregivers based on proximity to a patient. In some cases, the location information can be used to assist in documenting the performance of certain care protocols.

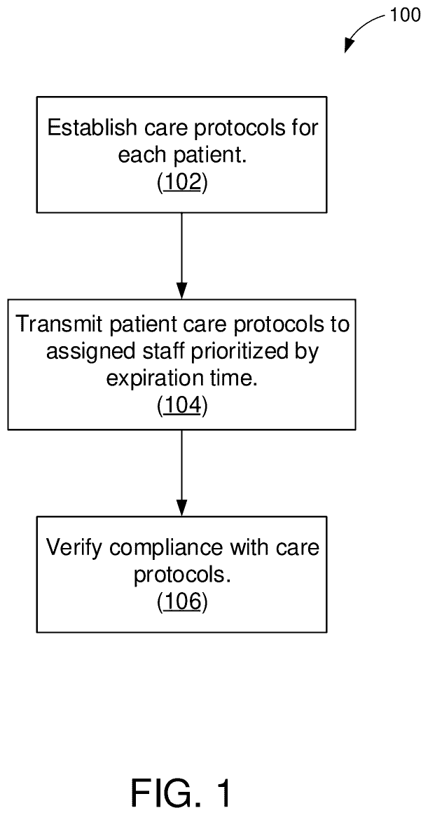

FIG. 1 is a flowchart of a method 100 for generating or creating patient care protocols and distributing the care protocols to caregiving staff. The method 100 describes in general terms operations associated with establishing, distributing, and verifying the performance of patient care protocols. Details associated with discrete operations are described in more detail below.

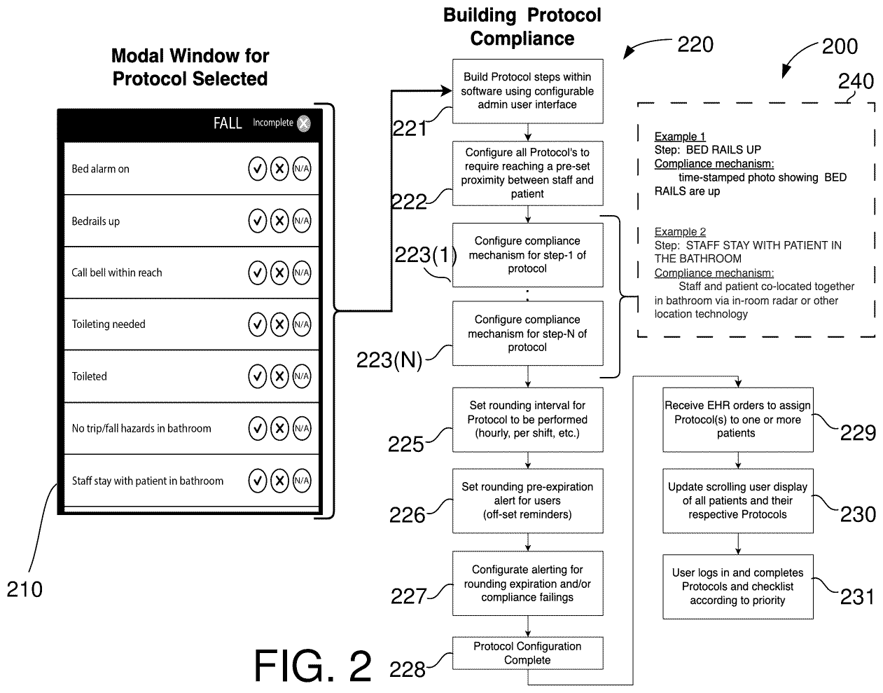

The method 100 begins in block 102 where care protocols are established for each patient. A patient can have any number of health care related conditions that can require actions or checks to ensure patient safety and improve patient outcomes. These actions or checks are sometimes referred to as care protocols. In block 102, elements that comprise a care protocol can be selected and assigned to a patient. In addition, a frequency of performance (hourly, daily, per nursing shift, and the like) can be associated with each care protocol element. Thus, for each patient, any number of care protocols can be established. Details associated with block 102 are described in more detail with respect to FIG. 2.

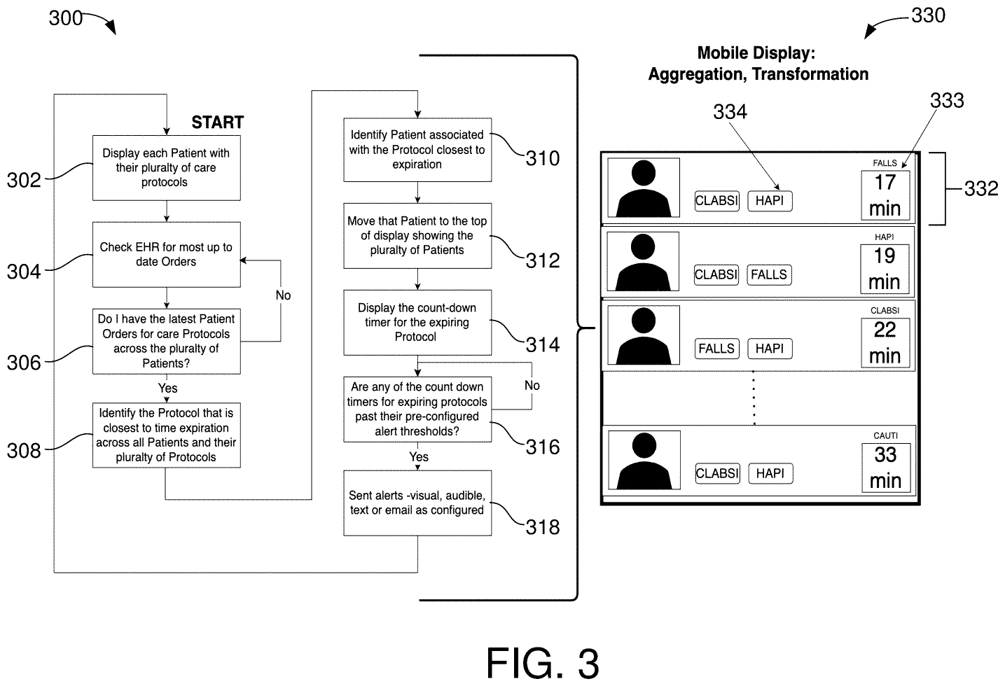

Next, in block 104 the patient care protocols are transmitted to assigned staff and prioritized by expiration time. Certain caregiving staff may be selected to care for and monitor a subset of patients. Thus, the patient care protocols for the subset of patients that are assigned to particular caregiving staff are transmitted to them. In some examples, the caregiving staff may have a mobile device (smart phone, tablet computer, smart watch, or the like) that receives one or more patient care protocols. The care protocols may be sorted by expiration time. That is, those protocols for patients that have upcoming due times (times that the protocols are to be performed) may be presented to the caregiving staff before protocols that have due times that are further in the future. In some cases, the caregiving staff need not be limited to only nurses, but can include other staff such as certified nursing assistants (CNAs), licensed vocational nurses (LVNs), or any other feasible staff members. Details associated with block 104 are described in more detail with respect to FIG. 3.

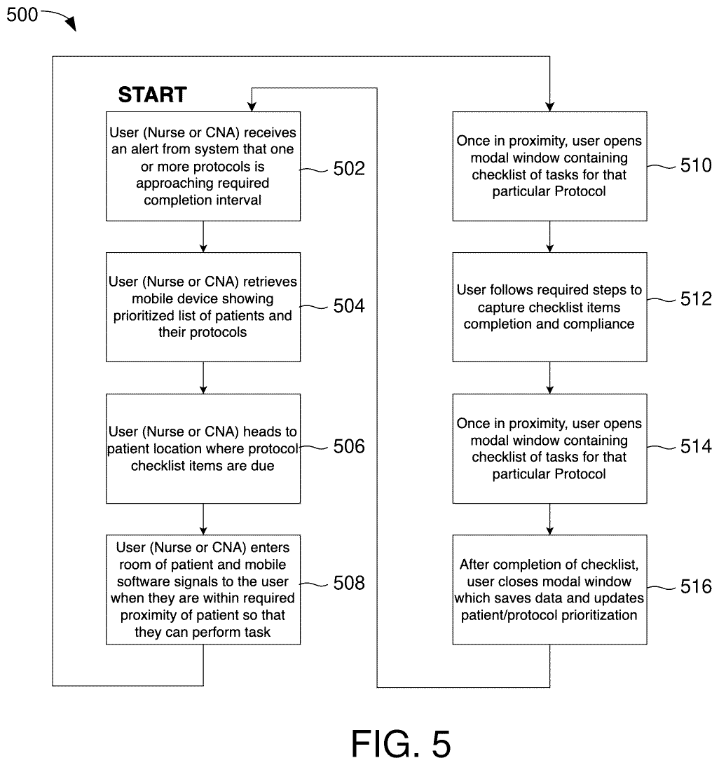

Next, in block 106 the performance of the care protocols is verified. Apparatuses, systems, and/or methods described herein can be used to document and verify that any number of health care protocols has been performed. Details associated with block 106 are described in more detail with respect to FIG. 5.

FIG. 2 shows an example user interface and flowchart for establishing or building care protocols for any number of patients. As a caregiver performs rounds to check on the status of a patient, a user interface 210 may be presented to the caregiver. The user interface 210 may be specific to a particular care protocol. In the example of FIG. 2, the user interface 210 shows elements of a fall prevention protocol. The user interface 210 can include a number of items (elements) in a check list format that are to be reviewed as part of the performance of the care protocol. The elements of any particular care protocol may be determined in accordance with method 220.

The method 220 shows operations that may be performed by an administrator (oftentimes a doctor) to determine elements that are to be associated with any care protocol. In block 221, a care protocol is built from individual elements (sometimes referred to as steps). These steps can be presented as choices to the administrator through a user interface (not shown). In this way, the administrator can customize a protocol for a particular healthcare setting.

Next, in block 222 each protocol can be assigned a preset (predetermined) “verification” proximity between the caregiver and the patient by the administrator. The proximity requirement helps ensure that the caregiver is reasonably proximate to the patient when the caregiver verifies that elements of the care protocol have been completed. In block 222, the administrator can select the verification proximity to be any feasible distance.

Next, in blocks 223(1)-223(N), the administrator determines the compliance mechanism for each element of the care protocol. For example, if a care protocol has N steps or elements, then the administrator can determine and assign N compliance steps to be performed by a caregiver to verify and document that the care protocol was performed. As shown in block 240, some compliance verification steps may include a receiving a time-stamped image (e.g., photograph, video, etc.) showing the completion of a step, an independent determination of the co-location of the patient and the caregiver, or the like.

Next, in block 225, the administrator determines or sets a rounding (time) interval for the care protocol. The rounding interval can refer to how frequent or often the care protocol is to be performed. Example rounding intervals can be based on time (hourly, or the like), per work shift, time of day, or any other feasible period or frequency. Using the rounding interval, an expiry time (expiration time) can be determined for a particular protocol. The expiry time refers a stipulated deadline for the protocol to be performed.

Next, in block 226, the administrator determines a pre-expiration alert period for the protocol. The pre-expiration alert period can be used to provide the caregiving staff a reminder that a particular protocol is due to be performed soon. Thus, based on the pre-expiration alert period, a reminder or alert can be sent to the caregiving staff to help ensure the timely performance of the care protocol.

Next, in block 227 user alerts are configured. The administrator can determine how frequently and what type of alert can be provided to the caregivers when a care protocol has not been performed within the pre-expiration alert period. In some cases, the type of user alert can include a visual, audible, or tactile (haptic) alert.

In block 228, the care protocol configuration is complete. The steps associated with blocks 221-227 may be repeated for each care protocol that is to be monitored, verified, and documented. Notably, the care protocols determined through blocks 221-227 may be used or assigned to any number of patients. For example, a care protocol for “falls” may be determined once, but used and assigned to any number of patients.

In block 229, the electronic health records (EHRs) of patients within the healthcare facility are received and appropriate care protocols are assigned to the patients. For example, the EHRs of the patients can be reviewed and a determination made regarding which care protocols should be assigned to which specific patients. For example, all patients having a central line inserted into their body can be assigned a Central Line Associated Blood Stream Infection (CLABSI) care protocol. Similarly, all patients having a risk of falling may be assigned a falls care protocol. In some examples, all of the patient care protocols can be collected and stored within a database. The database can be local, or remotely located and accessible through a network such as a cloud-based database.

In block 230, a user interface is updated for caregivers assigned to patients. Because certain caregivers are assigned to care for certain patients, the care protocols for those patients can be transmitted to those caregivers. For example, a caregiver may be assigned to care for patients A, B, and C. Information and data regarding the care protocols for patients A, B, and C can be transmitted to smart phones, tablet computes, and the like carried by the caregivers. The transmitted data can include aspects of the care protocol (rounding intervals, compliance verification steps, care steps), and also patient information from the patient's EHR (height, weight, patient photo), patient room number, current location, and the like.

Next, in block 231, a caregiver can log in, review outstanding protocols, and perform them. In some examples, the care protocols can be presented on a user interface accessible to the user. The user interface can list the care protocols in order of priority. In some cases, the order of priority can be based on an expiration time associated with each care protocol. In some examples, the caregiver can receive an alert to perform a care protocol before an expiration of time.

FIG. 3 shows an example care protocol prioritization method and an associated user interface. In some examples, a flowchart of a method 300 for prioritizing patient care protocols can begin in block 302 where each patient may be displayed on a user interface along with the patient's associated care protocols. Notably, a single patient may be associated with a one or more care protocols. The user interface may include a display on a smart phone, a tablet computer, a laptop, a smart watch, or any other feasible display.

Next, in block 304 the patient's EHR is analyzed or examined for doctor's orders that include any up-to-date care protocols that have been assigned to the patient. In block 306, if the latest doctor's orders are not available, then the method returns to block 304. The algorithm may determine that there exist other, more recent care protocols from the doctors. For example, The apparatuses and methods may automatically receive updates to orders (for protocols, etc.) from the patient's EHR, and/or may include logic that is configured so that when the system receives and order update, the method and apparatus may know what fields to update. This may be performed according to the Health Level 7 (HL7) standard.

In some examples, the operations of blocks 304 and 306 advantageously help ensure that any changes to doctor's orders for a patient are captured, particularly with respect to changes in care protocol. Even changes that are changed within a day, or even mid-shift.

Next, in block 308, the care protocol that is closest to expiry (that is, the care protocol that is to be performed next) is identified. Next, in block 310 the patient associated with the identified care protocol is identified.

Next, in block 312, the identified patient and care protocol is displayed on a user interface. In some examples, the identified patient and care protocol may be made prominent on the user interface by being displayed on the top of a list showing several care protocols. In some cases, the displayed patient care protocols can be arranged in order of time expiration. Thus, the care protocol that is closest to an expiration time is placed at the top of the user interface or list. Other patient care protocol are listed in descending order of expiration time.

Next, in block 314 a countdown time for the care protocol is displayed. The countdown time shows how close is the care protocol to the expiry time.

Next, in block 316 if there are no care protocols that have an expiry time less than a pre-expiration alert period, then the method returns to block 316. As described with respect to FIG. 2, the pre-expiration alert period can be used to provide the caregiving staff a reminder that a particular protocol is due to be performed soon.

On the other hand, if a care protocol has an expiry time less than the pre-expiration alert period, then in block 318 an alert is transmitted to the caregiver. The alert can include visible (pop-up windows, text messages, email), audible (sounds and noises), and/or tactile (haptic) content. The method returns to block 302.

FIG. 3 includes an example user interface 330. The user interface 330 can display several patients with their associated care protocols. Entry 332 is associated with a patient 333. Entry 332 also a count-down timer 333 for the care protocol closest to its expiration time. In the entry 332, a falls protocol is due to be performed in seventeen minutes. The entry 332 can also show additional care protocols 334 associated with the patient.

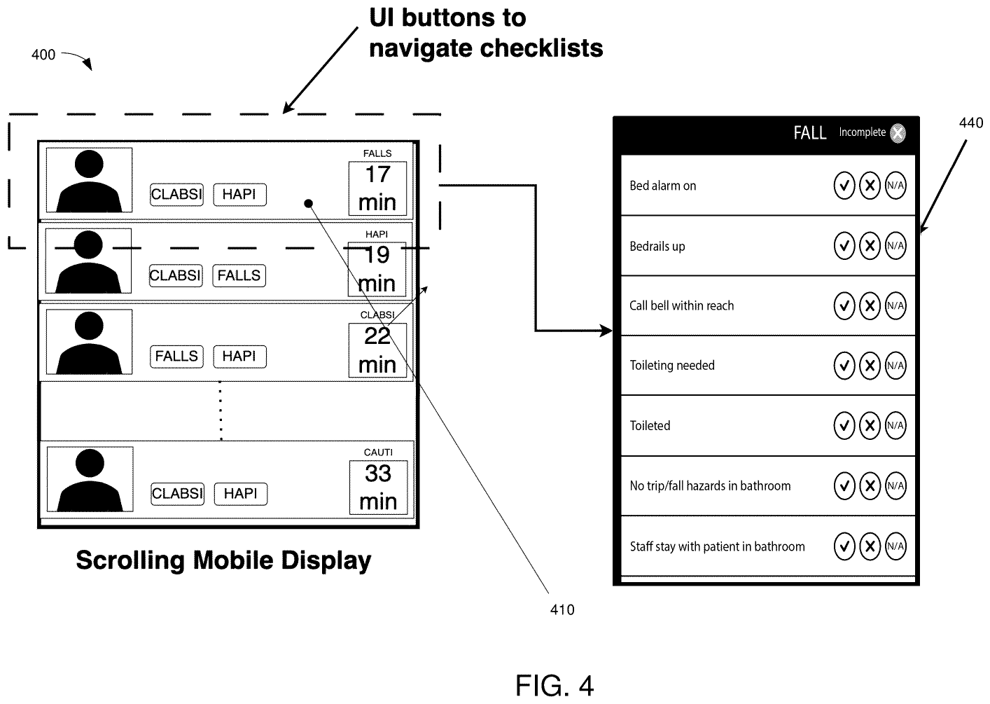

FIG. 4 shows various user interfaces to alert caregivers regarding the performance of care protocols. A first user interface 400 (which can be another example of the user interface 330 of FIG. 3) shows a prioritized (with respect to expiration time) list of patients and their respective care protocols. Entry 410 shows the care protocols for a particular patient and a countdown timer for the care protocol closes to expiration. In this example, a falls care protocol is due to be performed within seventeen minutes.

The first user interface 400 can be interactive. Thus, a caregiver can select entry 410 (with a finger press, mouse click, or the like) of the first user interface 400 and review details of the closest due care protocol. In this manner, when the caregiver interacts with entry 410, a second user interface 440 is displayed. Elements of the care protocol associated with entry 410 are displayed in the second user interface 440. In some examples, the caregiver can interact with the second user interface 440 to indicate and document that elements of the care protocol shown in the second user interface 440 are performed.

FIG. 5 is a flowchart showing an example method 500 for performing and verifying patient care protocols. Some examples may perform the operations described herein with additional operations, fewer operations, operations in a different order, operations in parallel, and some operations differently.

The method begins in block 502 where a user receives an alert that a care protocol for a patient is approaching a required completion interval. In some examples, a care protocol can be established or defined by an administrator as described herein with respect to FIG. 2. The completion interval (sometimes referred to as a rounding interval) can indicate how frequently a particular care protocol is to be performed. Approaching a required completion interval can refer to coming to the end of a countdown timer to when the care protocol is to be performed. A user can be any qualified caregiver and can include nurses, CNAs, LVNs, or any other feasible care personnel. In some examples, the alert can be a visual, audible, and or tactile alert.

Next, in block 504 the user can view a prioritized list of care protocols. The list can show patients and care protocols in descending order of completion or performance time. Thus, the prioritized list can list the care protocols in order of expiration time; the care protocols nearer the top of the list being closer to expiration than the care protocols nearer the bottom of the list. In this way, the user can easily determine the care protocol closest to expiration.

Next, in block 506 the user proceeds to the location of the patient whose care protocol is the soonest to expire. In some variations, a user interface can show the location of the patient.

Next, in block 508 the user is notified that the user is within a predetermined proximity (a predetermined distance) to the patient. When the user is within the predetermined proximity, then the user is close enough to the patient that he/she can perform various elements of the care protocol.

Next, in block 510 the user interacts with a user interface to display a checklist of one or more elements to be performed for the care protocol of the nearby patient. The checklist can advantageously remind the user of the elements or steps associated with a particular care protocol.

Next, in block 512 the user follows the steps displayed in the user interface with respect to block 510. The steps may include not only steps to complete the care protocol, but also steps or operations to verify that care protocol has been completed correctly and completely. In some cases the user may be instructed to take a picture to verify completion of at least an element of a care protocol. In another example, the location of the user and the patient can be verified and documents as corroboration that the user was nearby to the patient to perform an element of a care protocol.

Optionally, the step 510 of the user interacting with a user interface to display a checklist of one or more tasks to be performed for the care protocol of the nearby patient may be repeated or may be performed after capturing checklist items 512, as shown in block 514.

Next, in block 516 the user can close the user interface to save the verification data, mark this care protocol as complete, and update the list of prioritized care protocols. The method returns to block 502. In some cases, relevant verification data can be written to the appropriate patient EHRs. Writing verification data directly to the EHR can advantageously avoid documenting actions with paper, which may help avoid negative findings by regulatory bodies and/or payors. In some examples, the verification data can be stored in a database and be used to provide real-time compliance information.

The method 500 may be performed by any qualified personnel in the care staff. For example, a number of patients may be under the direct care of Nurse A. Nurse A may have CNA 1, CNA 2, and CNA 3 under her supervision. CNA 1, CNA 2, and/or CNA 3 can receive alerts and perform the indicated care protocols while Nurse A can receive status messages indicating that the care protocols have been performed. Thus, Nurse A is ensured that the protocols have been completed without having to be present with a CNA at each patient. In addition, the user interface can guide and inform the CNAs how to perform and document each care protocol.

In some aspects, the method 500 can optimize patient care workflow for task compliance. The method 500 monitors for the most up to date physician orders and any associated care protocols. Each patient may be displayed on a user interface (i.e., mobile, desktop, dashboard) along with the appropriate care protocols that have been assigned to that patient (i.e., Falls, CLABSI, etc.). Each care protocol can be pre-configured with count-down timers regarding how often those care protocols have to be performed (hourly, shift, day, etc.). The method 500 can continually search across all care protocols for all patients and displays that patient at the top of the user interface. For each patient, the method 500 can search across all of the count-down timers for all protocols associated with a particular patient. The count-down timer of that patient's next expiring protocol is displayed. As such, the patient having the protocol closest to expiration will always be displayed at the top followed by the patient with the 2^{nd} closest to expiration care protocol and so forth to create an ordered list.

Each care protocol may have pre-configured alerts for any user interfaces (visual, audible, tactile, etc.) as well as the ability to send text, email or other generalized for role/person specific. If expiration times are hit without the care protocol being completed a series of alerts can be sent to supervisory personnel for follow-up. Because the system is constantly updated, prioritized, and configured to alert across all patients, high levels of compliance can be achieved.

In some examples, if expiration times are exceeded without the care protocol being completed, a series of alerts can be sent to supervisory personnel for follow-up. Because the system is constantly updated, prioritized, and configured to alert across all patients, high levels of compliance can be achieved.

The workflow demonstrated in FIG. 5 can provide a significantly more efficient workflow for the caregiving staff. A nurse may be responsible for 4 patients and each one may have up to 4 care protocols. Each care protocol can typically have a checklist (care protocol) of up to 10 checklist items. CNA's, under nursing supervision, also perform care tasks associated with the care protocols. While staffing ratios vary, CNA to patient ratios typically range from 1:6 or 10 depending on the acuity of the unit. As such, CNA's may support multiple nurses with care protocol tasks associated with their patients. Because the method 500 creates visibility as to the status and completion of care protocols across all patients, no nurse-to-CNA trips are needed to verify whether tasks have been completed. Such a method 500 also allows for maximum flexibility in how nurses and CNA's can be used to support care protocol requirements. In some examples, any CNA could attend to any care protocol task.

One advantage of the method 500 is in its ability to automatically document whether checklist items have been completed as well as their compliance status. Currently, nurses and CNA's have to spend a substantial amount of their time manually documenting the completion of tasks. The method 500 can automatically capture and store checklist item completion, compliance status as well as quantifying, in many cases, the degree of completion and/or a need for care escalation.

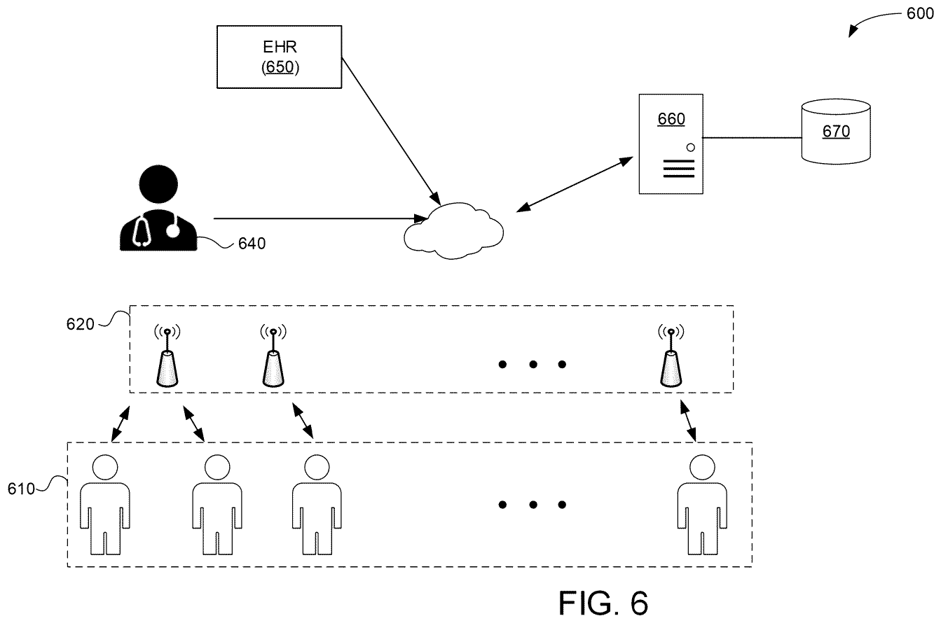

FIG. 6 shows an example system 600 for monitoring compliance with patient care protocols. The system 600 includes access points 620, a network 630, a computing device 660 and a datastore 670. The access points 620 can communicate with and locate users and patients 610. The users and patients 610 may have access to portable computing devices such as smart phones, tablet computer, smart watches, and, in some cases, other wearable computing devices. The access points 620 can be coupled to the computing device 660 through the network 630.

In some examples, the computing device 660 can receive or access patient's EHRs 650 and receive doctor input 640 to generate one or more patient care protocols. The patient care protocols can be transmitted to the users' portable computing devices (not shown). The portable computing devices can prioritize the care protocols based on their expiration time. The portable computing devices can alert the users and remind them of expiring care protocols that need to be performed. The users can use the portable computing devices to document and verify that the care protocols were performed timely and correctly. The verification data can be transmitted to the computing device 660. The datastore 670, coupled to the computing device 660 may be used to store the verification data, in addition to EHRs 650 and patient care protocols.

In some variations, the computing device 660 can determine the location of the users and patients 610 through various locationing procedures. Example locationing procedures can include using receive signal strength indicators (RSSI), angle of arrival (AoA), angle of departure (AoD), beamforming, GPS information, proximity sensor information, and the like.

In some examples, the computing device 660 can assign patient care protocols to patients based on patient information in the EHRs 650. The doctor 640 can determine patient care protocols and the computing device 660 can review patient EHRs 650 and assign the appropriate care protocols to the appropriate patients. In some examples, the system 600 can include software that organizes, prioritizes, and prompts care protocols for all the patients that may be in a hospital unit. The system 600 can incorporate a variety of mechanisms to enforce real-time compliance with any number of patient care requirements that have both physical and temporal requirements. The system 600 can also automatically document complex care tasks.

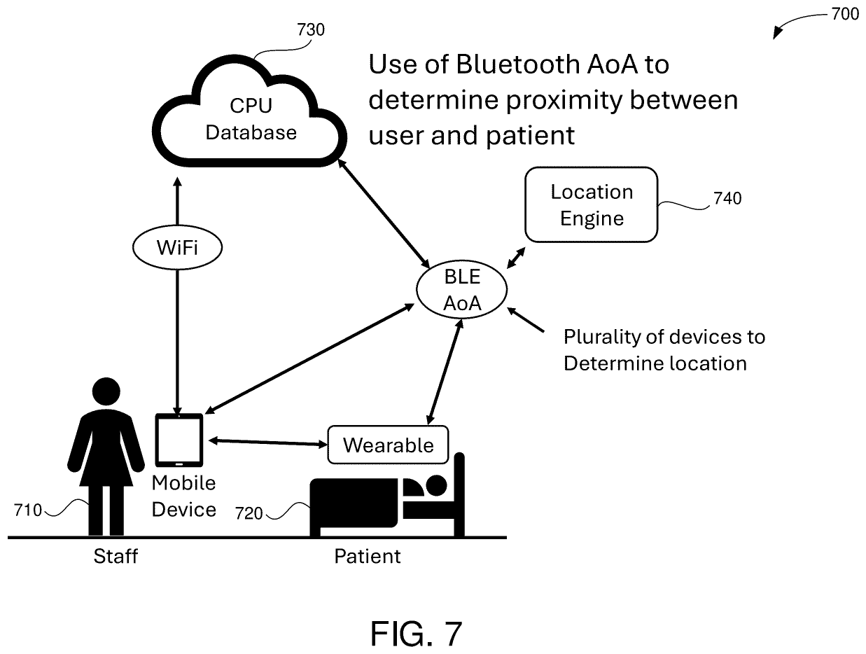

FIG. 7 shows a system 700 that can be wholly or partially included in the system 600 of FIG. 6. The system 700 can include a staff member 710, a patient 720, a CPU database 730, and a location engine 740. The system 700 can be used to provide care protocol information to the staff member 710 (a user), for the patient 720. In some examples, the CPU database 730 (also referred to herein as a computing device) can determine a location of the staff member 710 and the patient 720. The location engine 740, which may be included within or coupled to the CPU database 730, can use wireless signals from a mobile device (associated with the staff member 710) and a wearable device (associated with the patient 720) to determine their locations. For example, the location engine 740 can use Bluetooth angle of arrival information as well as Wi-Fi signal information to determine the location of the staff member 710 and the patient 720.

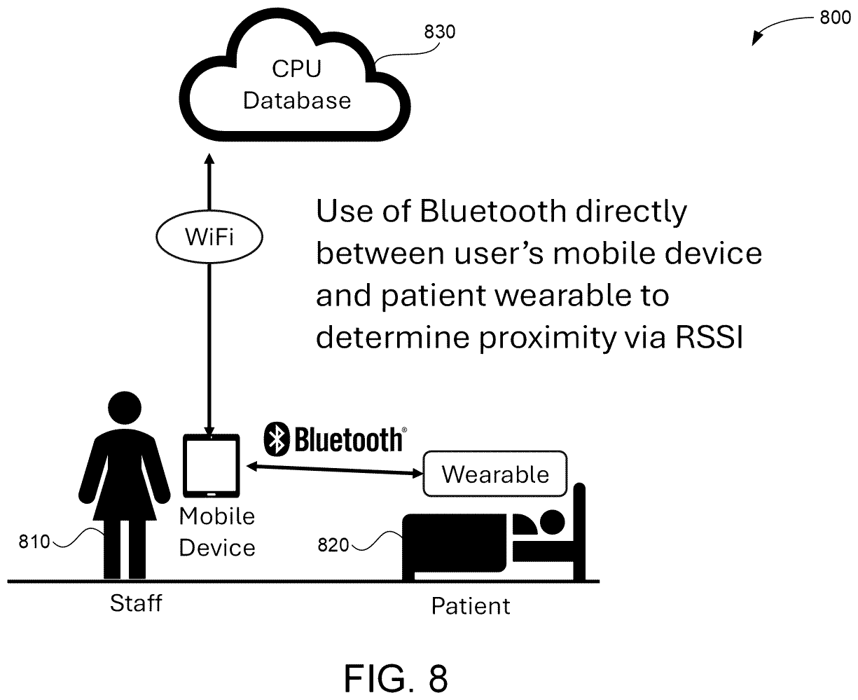

FIG. 8 shows a system 800 that can wholly or partially be included in the system 600 of FIG. 6. The system 800 can include a staff member 810, a patient 820, and a CPU database 830. In some examples, a mobile device associated with the staff 810 can determine a proximity to the patient 820. The patient 820 can wear a Bluetooth capable wearable device. The mobile device can determine Bluetooth RSSI information associated with the wearable device. Based on the RSSI, the mobile device can confirm the location of the patient 820 and provide this location information to the CPU database 830. In response, the CPU database 830 can send an alert to the staff member 810 to perform a care protocol for the patient 820.

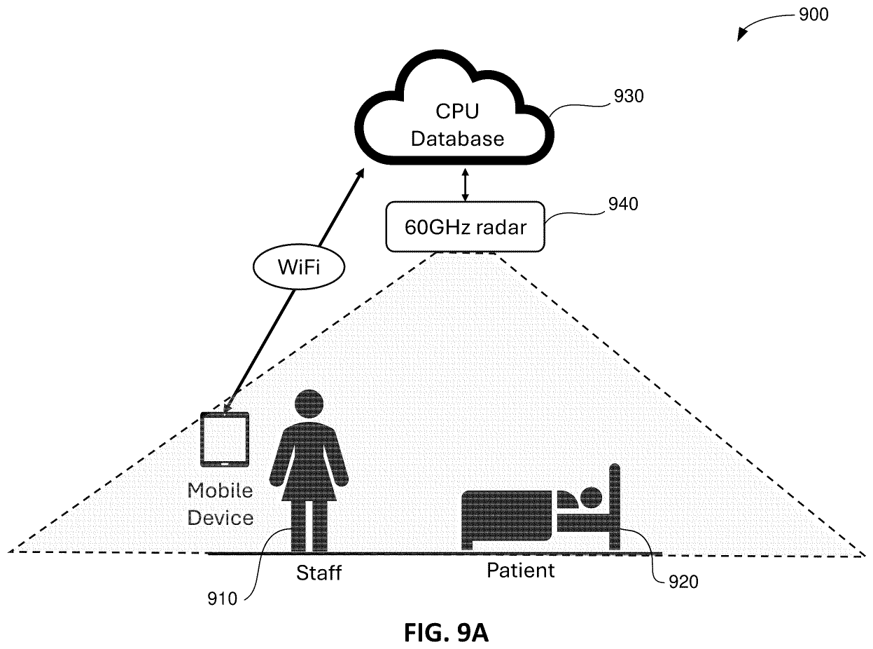

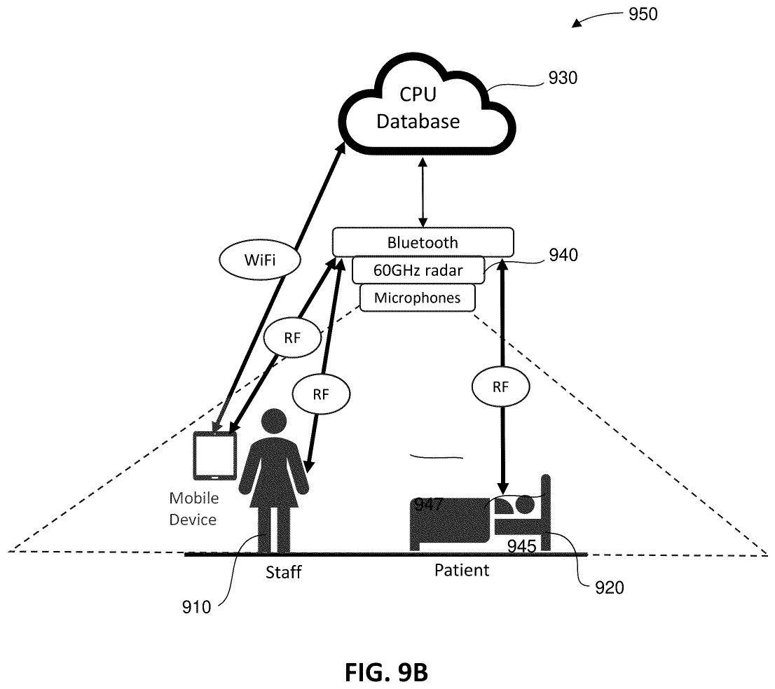

FIG. 9A shows a system 900 that can wholly or partially be included in the system 600 of FIG. 6. The system 900 can include a staff member 910, a patient 920, a CPU database 930 and a millimeter wave (mmWave) radar device 940. The mmWave radar device 940 can independently determine the location of the staff member 910 and the patient 920 and provide the location information to the CPU database 930.

FIG. 9B shows an example of another sub-system 950 similar that shown in FIG. 9A that can wholly or partially be included in an apparatus 600 similar, but not limited to, that shown in FIG. 6. The system 900 can also include a staff member 910, a patient 920, a CPU database 930 and a millimeter wave (mmWave) radar device 940. The mmWave radar device 940 can independently determine the location of the staff member 910 and the patient 920 and provide the location information to the CPU database 930. FIG. 9B also includes a Bluetooth receiver/transmitter 945, and a microphone 647, as described herein. In this example, mmWave radar may be used to determine proximity between user and patient, especially at distances that exceed the accuracy capabilities of other location technologies. Bluetooth (RSSI, AoA, AoD) may be used to determine proximity between user and patient where the Bluetooth device is integrated with the radar device. Any of these apparatuses may include one or more microphones incorporated into the device could also be used to capture signature sounds associated with a compliance function such as bedrail movement, toileting, etc.

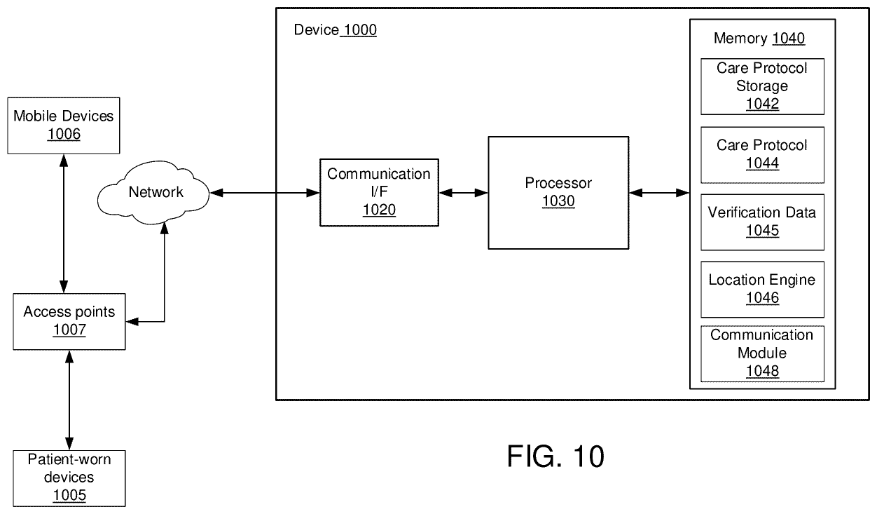

FIG. 10 shows a block diagram of a device 1000 that may perform any of the methods or procedures described herein. The device 1000 may include a communication interface 1020, a processor 1030, and a memory 1040.

The communication interface 1020, which may be coupled to a network (such as the Internet) and to the processor 1030, may transmit signals to and receive signals from other wired or wireless devices, including remote (e.g., cloud-based) storage devices, cameras, processors, compute nodes, processing nodes, computers, mobile devices (e.g., cellular phones, tablet computers and the like) mobile devices 1006, access points 1007, and patient-worn devices 1005. In some examples, the communication interface 1020 may include wired (e.g., serial, ethernet, or the like) and/or wireless (Bluetooth, Wi-Fi, cellular, or the like) transceivers that may communicate with any other feasible device through any feasible network.

The processor 1030, which is also coupled to the memory 1040, may be any one or more suitable processors capable of executing scripts or instructions of one or more software programs stored in the device 1000 (such as within memory 1040).

The memory 1040 may include care protocol storage 1042 that may be used to locally store patient care protocols. In some examples, a doctor can determine and store different patient care protocols within the care protocol storage 1042.

The memory 1040 may include verification data storage 1045 to locally store verification data collected from users.

The memory 1040 may include a non-transitory computer-readable storage medium (e.g., one or more nonvolatile memory elements, such as EPROM, EEPROM, Flash memory, a hard drive, etc.) that may store the following software modules:
a care protocol module 1042 to enable the performance of care protocols for different patients;
a location engine 1046 to determine a location of an object within the 3D point cloud image; and
a communication module 1048 to communicate with other devices.
   Each software module includes program instructions that, when executed by the processor 1030, may cause the device 1000 to perform the corresponding function(s). Thus, the non-transitory computer-readable storage medium of memory 1040 may include instructions for performing all or a portion of the operations described herein.

The processor 1030 may execute the protocol module 1042 to determine, prioritize, and alert caregivers to perform one or more care protocols. Execution of the protocol module 1042 can also enable the collection of care protocol verification data that is stored in the verification data storage 1045. Execution of the protocol module 1042 can cause the processor 1030 to perform any of the operations described with respect to FIGS. 1-9.

The processor 1030 may execute the location engine 1046 to determine the location of patients and staff within a monitoring region. For example, execution of the location engine 1046 may cause the processor 1030 to determine a location of a patient, or caregiver within the monitoring region.

The processor 1030 may execute the communication module 1048 to communicate with any other feasible devices. For example, execution of the communication module 1048 may enable the device 1000 to communicate via cellular networks conforming to any of the LTE standards promulgated by the 3^{rd} Generation Partnership Project (3GPP) working group, Wi-Fi networks conforming to any of the IEEE 802.11 standards, Bluetooth protocols put forth by the Bluetooth Special Interest Group (SIG), Ethernet protocols, or the like. In some embodiments, execution of the communication module 1048 may enable the device 1000 to communicate with other remote computing devices not shown. In some other embodiments, execution of the communication module 1048 may implement encryption and/or decryption procedures.

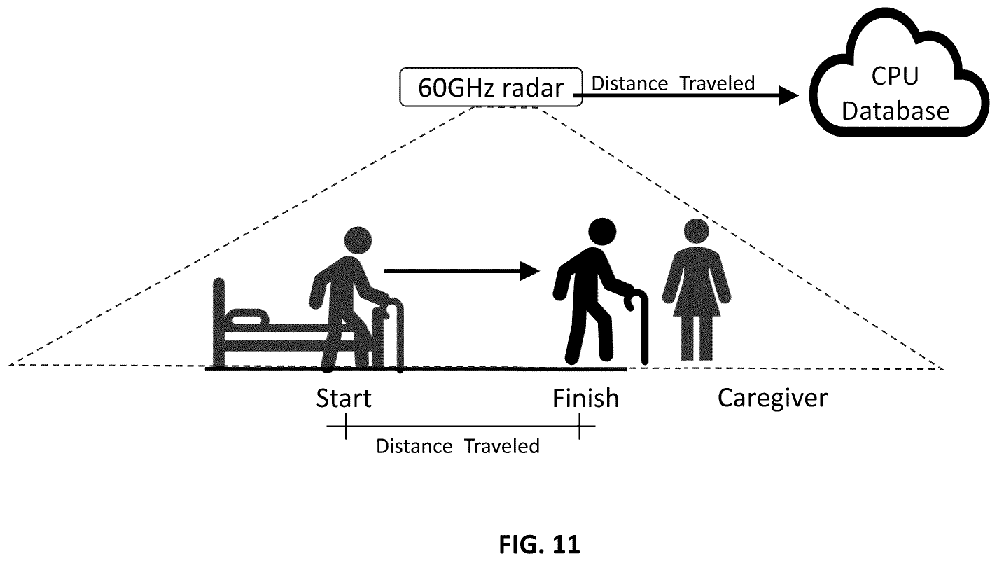

FIG. 11 schematically illustrates another example of the use of a subsystem that may be used with any of the apparatuses described herein. In FIG. 11 the apparatus includes a radar (e.g., a 60 GHz radar) from which distance traveled by a subject, e.g., patient, caregiver (which may be monitored by the 60 GHz radar) and used to determine the distance (e.g., via the remote, e.g., off-site, as shown. In this example, radar may be used to precisely measure the distance or any kind of ambulation a patient has traveled—Useful for short distances—and automatically sending that data to a medical record. For example right after surgery, such as, but not limited to, abdominal surgery.

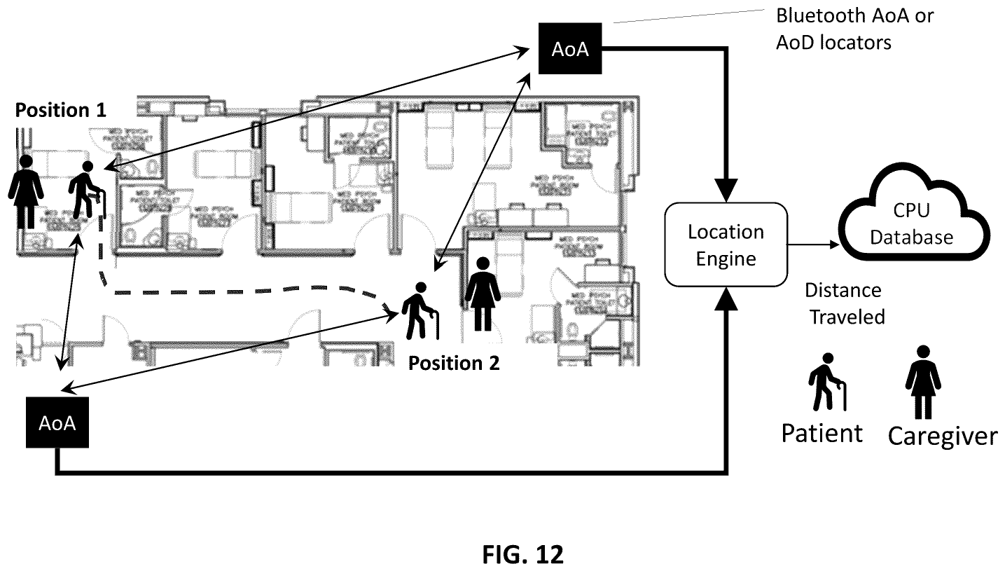

FIG. 12 schematically illustrates another example of the use of a subsystem that may be used with any of the apparatuses described herein. In FIG. 12 either or both Bluetooth AoA or AoD technology may be used to measure distance traveled by patient ambulation as well whether staff person is within proximity of the patient at all times (proximity compliance). AoA and AoD are precision technologies that are capable of accurately capturing macro distances/location of patient ambulation. The distance traveled could be part of a ERAS (Enhanced Recovery After Surgery) protocol where the patient is required to progressively walk longer distances on the hospital unit to indicate readiness to go home. This distance can be automatically captured by the invention as part of the Protocol System.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations, or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached, or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise,” and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components, or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method, the method comprising:
receiving or accessing, in one or more processors, one or more hospital or physician orders for each patient of a set of patients within a healthcare location;
determining, by the one or more processors, a patient care protocol for each of the hospital or physician orders for each patient, wherein each patient care protocol comprises: one or more steps to be performed by a caregiver, one or more timers associated with the one or more steps, and one or more compliance indicators for each of the one or more steps, wherein the compliance indicators each include a proximity indicator indicating that a patient of the set of patients is within a predetermined distance of the caregiver and a time-stamped image providing visual confirmation of compliance of a step of the patient care protocol;
displaying, on a user interface, a time-ordered list of the patient care protocols for the set of patients, with a countdown time associated with each patient care protocol in the time-ordered list;
receiving a radio frequency (RF) signal from a device of the patient and determining a location of the patient based on the received RF signal;
confirming compliance, by the one or more processors, with each step of the patient care protocol of the time-ordered list of patient care protocols based on receipt of the proximity indicator and the time-stamped image by the one or more processors, the proximity indicator associated with the patient care protocol, the determined location of the patient based on the received RF signal, and one or more times associated with the one or more steps; and
outputting an indication that all or some of the one or more steps of the patient care protocol have been completed based on the confirmed compliance.

2. The method of claim 1, wherein detecting proximity data comprises detecting proximity data using a mobile device held by the caregiver or a device worn by the caregiver.

3. The method of claim 1, wherein determining the location of the patient further comprises detecting proximity using one or more of: a LiDAR signal, a radar signal, an ultrasound signal and/or a radio frequency (RF) signal between a caregiver device and a patient worn device.

4. The method of claim 1, wherein determining the location of the patient comprises detecting proximity using one or more of: a LiDAR signal, a radar signal, an ultrasound signal and/or a radio frequency (RF) signal between a caregiver device and a patient worn device and an RF device that is integrated with a radar, LiDAR or ultrasound device.

5. The method of claim 3, wherein the RF signal comprises an RF received signal strength indicator (RSSI) from directly between devices carried by the caregiver and the patient.

6. The method of claim 3, wherein the RF signal between the caregiver device and the patient worn device comprise one of: an RF angle of arrival (AoA) and/or angle of departure (AoD) and/or a time of flight scheme, from a caregiver device and a patient-worn device.

7. The method of claim 3, wherein the LiDAR, radar or ultrasound creates a two-or three-dimensional physical representation model to gauge proximity by comparing temporal changes in the model based on distance and/or motion.

8. The method of claim 1, wherein identifying the patient care protocol associated with each patient of the set of patients from the physician orders further comprises determining the one or more times associated with the one or more steps of each patient care protocol.

9. The method of claim 1, further wherein each patient protocol comprises one or more compliance indicators associated with the one or more steps.

10. The method of claim 9, wherein confirming compliance with the patient care protocol of the time-ordered list of patient care protocols is based on the proximity data, the proximity indicator associated with the patient care protocol, the one or more times associated with the one or more steps and the one or more compliance indicators.

11. The method of claim 1, wherein identifying the patient care protocol comprises identifying from a database of care protocols.

12. The method of claim 1, wherein receiving or accessing the one or more physician orders for each patient of a set of patients within a healthcare location comprises receiving or accessing patient electronic health care records (EHR).

13. The method of claim 1, further comprising identifying the set of patients within the healthcare location.

14. The method of claim 1, wherein displaying comprises displaying the time-ordered list of the patient care protocols for the set of patients on a user interface of a mobile device.

15. The method of claim 1, wherein outputting the indication that all or some of the one or more steps of the patient care protocol have been completed based on the confirmed compliance comprises transmitting to a remote device.

16. The method of claim 1, wherein outputting comprises removing a completed patient care protocol from the time-ordered list of the patient care protocols.

17. The method of claim 1, wherein the time-stamped includes a time-stamped photograph.

18. The method of claim 17, wherein confirming compliance comprises identifying, from the photograph, that at least one of the one or more steps has been completed.

19. The method of claim 17, wherein confirming compliance comprises using a trained machine learning agent to identify from the photograph, that at least one of the one or more steps has been completed.

20. A method, the method comprising:
receiving or accessing, in a processor, one or more hospital and/or physician orders for each patient of a set of patients within a healthcare location;
determining, by the processor, a patient care protocol for each of the hospital and/or physician orders for each patient, wherein each patient care protocol comprises: one or more steps to be performed by a caregiver, one or more timers associated with the one or more steps, and one or more compliance indicators for each of the one or more steps, wherein the compliance indicators each include a proximity indicator indicating that a patient of the set of patients is within a predetermined distance of the caregiver and a time-stamped image providing visual confirmation of compliance of a step of the patient care protocol;
displaying, on a user interface of a caregiver device, a time-ordered list of the patient care protocols for the set of patients, with a countdown time associated with each patient care protocol in the time-ordered list;
receiving a radio frequency (RF) signal from a device of the patient and determining a location of the patient based on the received RF signal;
confirming compliance with a patient care protocol of the time-ordered list of patient care protocols based on the proximity data, the proximity indicator associated with the patient care protocol, the determined location of the patient based on the received RF signal, and one or more times associated with the one or more steps; and
outputting an indication that all or some of the one or more steps of the patient care protocol have been completed based on the confirmed compliance and updating the user interface of the caregiver device to reflect partial and/or complete completion of the patient care protocol.
